(19) United States
(12) Patent Application Publication
(10) Pub. No.: US 2005/0169846 A1
(43) Pub. Date: Aug. 4, 2005

(54) Enhancement of fragrance release from candles

(76) Inventor: Bart Kennington, Layton, UT (US)

Correspondence Address: Mr. Lynn G. Foster, 602 E. 300 S., Salt Lake City, UT, 84102, US

(21) Appl. No.: 10/770,330

(22) Filed: Jan. 31, 2004

(51) Int. Cl. A61L 9/02; A61K 9/72

(52) U.S. Cl. 424/40; 424/76.1

(57) ABSTRACT

Combinations, assemblies and methods for releasing fragrance from candles and like objects are disclosed, which utilize a heat conductive core or element embedded in the material from which the containerized candle or like item is made whereby heat is applied to the container is distributed to the material along the core to facilitate more rapid and more uniform melting, while reducing the risk of an explosion due to uneven heating.

### FIELD OF THE INVENTION

The present invention relates generally to emission of fragrance from a candle or the like and, more particularly, to novel combinations, assemblies and methods by which a normally solid candle or like item in a receptacle or container is melted by heat applied through the receptacle directly to adjacent material comprising the candle or like item and indirectly along a heat conducting element embedded in the material to more remote material locations.

### BACKGROUND

Perfumed candles and like fragrance releasing items in a solid state at room temperature are typically placed in a container, such as an open top glass vessel. Fragrance is emitted from the material comprising the candle or like item by either burning a wick to melt the top of the candle, or externally heating the bottom of the container to melt to lower portions of the candle or like object.

Wick burning releases pollutants which to some extent mutes the perfume fragrance also being released and, with some candle compositions, can be harmful to health.

Non-uniform heating of the material from which the candle or like item is made can result in an explosion, risking human injury and destroying the assembly including the container.

### BRIEF SUMMARY AND OBJECTS OF THE INVENTION

In brief summary, the present invention overcomes or alleviates problems pertaining to the release of fragrance from and/or the melting of candles and the like. A heat conductive core or element is embedded in the material from which the containerized candle or like item is made, whereby heat applied to the container is distributed to the material along the core to facilitate more rapid and more uniform melting, while reducing the risk of an explosion due to uneven heating.

With the foregoing in mind, it is a primary object to overcome or alleviate problems of the past pertaining to release of fragrance from and/or the melting of candles and like items.

Another valuable object is the provision of novel combinations or assemblies and unique methods for releasing fragrance from and/or melting candles and like items.

A further paramount object is the provision of novel combinations, assemblies and method utilizing a heat conductive core or element embedded in a containerized candle or like item to better distribute heat applied to the container for more rapid melting while reducing the risk of an explosion.

These and other objects and features of the present invention will be apparent from the detailed description taken with reference to accompanying drawings.

### BRIEF DESCRIPTION OF THE DRAWINGS

FIG. 1 is a perspective of a heat conductive core about to be inserted into a containerized candle;

FIG. 2 is a vertical cross section of the containerized candle of FIG. 1 with the heat conductive core embedded therein;

FIG. 3 is a vertical cross section similar to FIG. 2, but with the heat conductive core inverted;

FIGS. 4 through 7 are front elevation views of some representations for the container for receiving the candle or like item; and

FIGS. 8 through 13 are front elevation views of some representative heat conductive cores which may be embedded in the candle or like item.

### DETAILED DESCRIPTION OF THE ILLUSTRATED EMBODIMENTS

Reference is now made to the drawing wherein like numerals are used to designate like parts throughout. The present invention relates generally to emission of fragrance from a material used to form candles or like objects and comprises combinations, assemblies and methods by which a normally solid candle or like object in a receptacle, vessel or container is melted by heat applied through the bottom of the receptacle directly to the material comprising the candle. The heat also is communicated to a heat conducting element embedded in the material which conducts the heat to locations remote from the bottom of the receptacle to accommodate complete and more rapid melting of the material in a relatively short time, while reducing the risk of an explosion due to uneven heating.

Typically, the vessel is formed as one piece and comprised of glass so that the material from which the candle is made is visually observable through the vessel. While it is preferred that fragrance emission is limited to melting, if desired, where a candle is the subject of the melting process, the wick thereof may be caused to burn at the same time. Because candle burning producing emissions of combustion, ordinarily in confined locations, such as homes, users may prefer melting alone over simultaneous wick burning and melting. In this way, the fragrance is made available without potentially harmful releases of pollutants due to combustion.

While any source of low temperature heat may be utilized to melt the candle or like object in the vessel, one suitable approach is to use a mug warmer placed beneath the vessel. Typically, mug warmers have on and off positions so that when the fragrance of the candle or like object is no longer desired, the user simply shuts the warmer off, allowing the material from which the candle or like object is formed to return from a liquid state to a solid state. While the fragrance emission technique of the present invention may utilize a candle with a wick because of ready availability, there is no absolute requirement that the object to be melted be a candle nor that a wick be part of the object melted.

There is no constraint on the type of fragrance to be emitted, nor on the shape of the vessel, candle or heat-conductive core or element embedded in the material of which the candle or like object is comprised. The present invention accommodates either insertion of the heat-conductive core after formation of the candle or like object, or placement of the heat-conductive core or element in the material at the time the candle or like object is placed in the vessel.

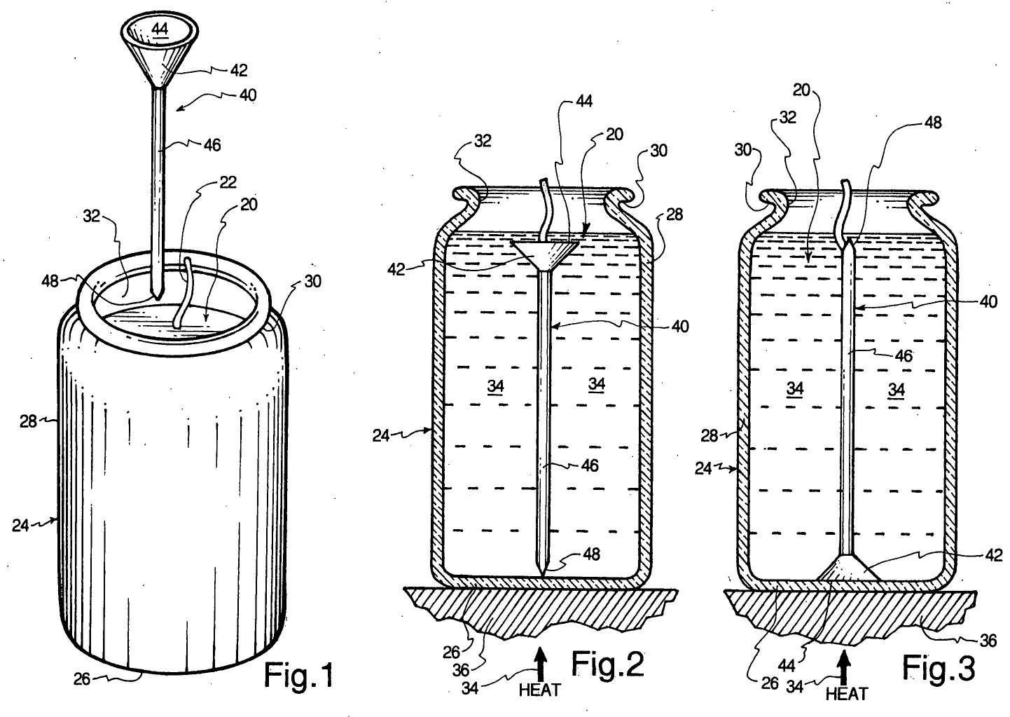

Attention is directed to FIG. 1, which illustrate a pre-existing candle, generally designated 20 comprising a wick 22 wherein the candle is encased by or confined in a vessel or container, generally designated 24. The vessel 24 is illustrated as being formed as one piece so as to comprise a bottom wall 26, an annular side wall 28, a necked down top 30 and an opening 32 through which fragrance from the perfumed material 34 of the candle or like object 20 escapes from the vessel 24 upon the application of heat 34 (FIG. 2) from a hot pad, mug warmer or the like source 36.

Heretofore, the process of melting the material 34 has been slow and non-uniform, sometimes causing the material 34 to explode, shattering the vessel 24. To reduce the risk associated with this problem, the present invention utilizes a heat-conductive core or element, generally designated 40. While any suitable heating element shape may be used, the shape of element 40 depicted in FIG. 1 comprises a conical head 42 having a flat surface 44 to accommodate application of force, as explained hereinafter. The conical head 42 merges with a slender rod or shaft 46 ending in a pointed distal tip of 48. Any suitable heat-conductive material may be used.

When the candle 20 is preformed and in a solid state, the heat-conductive core or element 40 is inserted by placing it generally in the position illustrated in FIG. 1 and, thereafter, inserting the same, by a manual pushing force applied to surface 44, into the center of the solid material 34 from which the candle or like object 20 is formed. This places the heat-conductive element generally in the position illustrated in FIG. 2. As a consequence, upon the application of heat 34 from low temperature heater 36, the heat passing through the bottom wall 26 is communicated to the rod 46 and thence to the head 42. The heat is distributed in a somewhat even fashion centrally along the full length of the material 34, causing the material 34 to melt more rapidly and more uniformly, producing fragrance in larger quantities in a shorter interval of time and also reducing, if not eliminating, the risk that the material 34 will explode due to uneven heating.

Once the material 34 is in a liquid state, the user may invert the heat-conductive element 40 by manually removing it from the melted material 34, inverting it and returning it to the melted material 34 into the position shown in FIG. 3. To prevent injury, pliers or the like may be used. This accommodates more rapid transfer of heat because of the larger mass at the head 42, now located at the bottom. It also increases the stability of the heat-conductive core 40 since the surface 42 reliably engages the bottom surface 26 of the vessel 24 to retain the heat-conductive element 40 in a secure central position, as illustrated in FIG. 3.

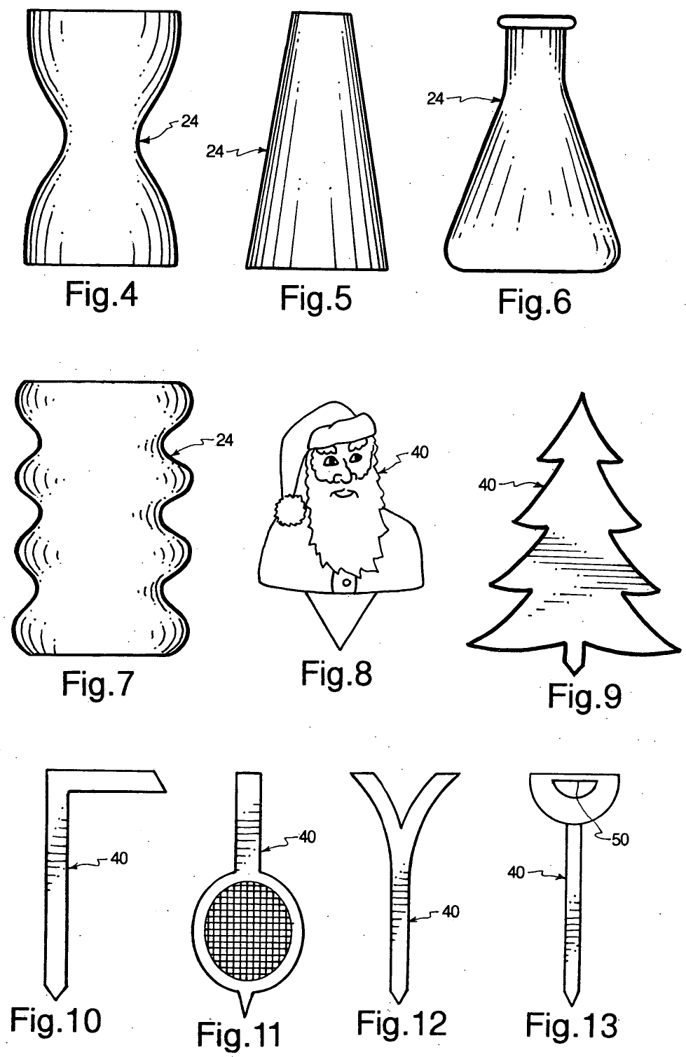

Reference is now made to FIGS. 4-7, which depict representative configurations for the vessel 24. In short, container, encasement or vessel 24 may be of any desired configuration, without departing from the substance of the present invention.

Likewise, the heat-conductive element 40, as shown in FIGS. 8-13 may comprise any of several configurations. These configurations are merely representative and may be entirely functional or may be decorative and functional, so long as they are heat-conductive so as to more uniformly heat the material 34. The heat-conductive element 40 of FIG. 13 comprises an aperture 50 by which a hook or the like can be used to remove the heat-conductive element when it is inverted from a position similar to FIG. 2 to a position similar to FIG. 3.

The invention may be embodied in other specific forms without departing from the spirit of the central characteristics thereof. The present embodiments therefore to be considered in all respects as illustrative and not restrictive, the scope of the invention being indicated by the appended claims rather than by the foregoing description, and all changes which come within the meaning and range of equivalency of the claims are therefore intended to be embraced therein.

1. A combination comprising:
a container;
a fragrance releasing material disposed in the container, the material being solid at room temperature but having a low melting point;
a heat-conductive core embedded or embeddable in the material to distribute heat applied to the material by which the fragrance is released.

2. A fragrance releasing combination comprising;
a vessel;
a quantity of fragrance-bearing material disposed in the vessel, the states of which is transformed from solid to liquid upon the application of low level heat;
an element placeable in the material within the vessel to distribute said heat more uniformly throughout the material.

3. The combination according to claim 1 further comprising a device by which heat is delivered to the material and the element through the container.

4. A fragrance emitting assembly comprising:
a candle comprised of wax-like fragrance containing material which is in a solid state at room temperature;
an impervious encasement containing the material;
a heat conductive element disposed within the material of which the candle is comprised;
whereby upon the application of heat to candle and the element through the encasement, the element distributes the heat to enhance melting and release of the fragrance from the material.

5. A method of releasing a fragrance from a normally solid fragrance bearing material disposed in a receptacle, comprising the acts of:
disposing a heat-conductive material within the material within the receptacle;
applying heat from an external source at a site across the vessel to the material and to the element;
applying heat from the element to the material remote from the external heat source.

6. A method of emitting a fragrance comprising the acts of:
transforming a solid material in a container to a liquid causing release of a fragrant odor by application of heat to the container and distributing the heat along a heat-conductive element disposed within the material.

7. A method of emitting a fragrance according to claim 6 further comprising the acts of removing the element from the liquid material, altering the orientation of the element and returning the reoriented element to the material.

8. A method of melting a candle without combustion, comprising the acts of combining a heat conductor and a candle, with or without a wick, in a container.
heating the container and the conductor to locally melt the candle and to distribute heat along the conductor to melt the candle away from the locally melt location.
